(19) United States
(12) Patent Application Publication
(10) Pub. No.: US 2025/0288991 A1
(43) Pub. Date: Sep. 18, 2025

(54) SWAB ASSEMBLIES AND ASSAY SYSTEMS

(71) Applicant: 3EO HEALTH INC., Beverly, MA (US)

(72) Inventors: Dave BRADBURY, Beverly, MA (US); Thomas SCHAUS, Beverly, MA (US); Peter HONKANEN, Beverly, MA (US)

(21) Appl. No.: 18/859,918

(22) PCT Filed: Apr. 26, 2023
(86) PCT No.: PCT/US2023/020008

§ 371 Date: Oct. 24, 2024

Related U.S. Application Data
(60) Provisional application No. 63/336,876, filed on Apr. 29, 2022.

(51) Int. Cl. B01L 3/00; A61B 10/00

(52) U.S. Cl. CPC B01L 3/502715; A61B 10/0045; B01L 3/5029; B01L 2200/027; B01L 2300/044; B01L 2300/0672; B01L 2300/069; B01L 2300/0832; B01L 2300/0858

(57) ABSTRACT

A swab assembly includes a swab device and a metering device, wherein the swab device extends through an interior of the metering device. An assay system configured to receive a swab device includes a generally cylindrical body, an open window extending through a side wall of the generally cylindrical body, and a locking tab extending from the generally cylindrical body partially through the open window, wherein the locking tab is configured to engage with the swab device to prevent retraction of the swab device out of the assay system.

### BACKGROUND

### Technical Field

The present disclosure relates generally to swab assemblies, including nasal swab assemblies for collecting biological samples, and to assay systems, including systems that can receive such swab assemblies.

### Description of the Related Art

A wide variety of swab and assay devices are commercially available. Nevertheless, there is room for improvement in such devices, including with respect to safety and effectiveness.

### BRIEF SUMMARY

A swab assembly may be summarized as comprising: a swab device; and a metering device; wherein the swab device extends through an interior of the metering device. The swab device may be made of a single monolithic piece of injection-molded plastic. The swab device may have a sample-collection tip end portion and a plurality of grooves formed in an outer surface of the sample-collection tip end portion. The plurality of grooves may include four grooves that are equally spaced apart from one another circumferentially about the outer surface of the sample-collection tip end portion. Each of the grooves may be linear and aligned with a central longitudinal axis of the swab device. Each of the grooves may be longer than a distance from the groove to a terminal tip end of the swab device. Each of the grooves may have a width of about 0.5 mm, a depth of about 0.34 mm, and a length of about 4.52 mm. The metering device may include an annular blade portion engaged with an outer surface of a sample-collection tip end portion of the swab device. The annular blade portion may be engaged with the outer surface of the sample-collection tip end portion of the swab device in an interference fit.

An assay system configured to receive a swab device may be summarized as comprising: a generally cylindrical body; an open window extending through a side wall of the generally cylindrical body; and a locking tab extending from the generally cylindrical body partially through the open window; wherein the locking tab is configured to engage with the swab device to prevent retraction of the swab device out of the assay system. The locking tab may extend along an axis substantially aligned with a central longitudinal axis of the assay system and may extend at least partially inward into an interior of the generally cylindrical body. The system may further comprise a first foil; wherein, when the swab device is inserted into the assay system, the swab device pierces the first foil. The system may further comprise a second foil; wherein the window is configured to receive a tool configured to interact with the swab device while the swab device is inside the generally cylindrical body to move the swab device and wherein, when the swab device is moved by the tool, the swab device pierces the second foil. The tool may be configured to be controlled by a stepper motor.

### BRIEF DESCRIPTION OF THE SEVERAL VIEWS OF THE DRAWINGS

FIG. 1 illustrates a perspective view of a swab assembly.

FIG. 2 illustrates a cross-sectional view of the swab assembly of FIG. 1.

FIG. 3 illustrates a perspective view of a metering device of the swab assembly of FIG. 1.

FIG. 4 illustrates a perspective view of a swab device of the swab assembly of FIG. 1.

FIG. 5 illustrates a first side view of a swab tip of the swab device of FIG. 4.

FIG. 6 illustrates a second side view, opposite to the first side view, of the swab tip of the swab device of FIG. 4.

FIG. 7 illustrates a perspective view of a portion of an assay system configured to receive the swab device of FIG. 4.

FIG. 8 illustrates another perspective view of the portion of the assay system of FIG. 7.

FIG. 9 illustrates a perspective view of some components of the portion of the assay system of FIG. 7, including a first foil or membrane.

FIG. 10 illustrates the same perspective view of FIG. 9 of the same components of the portion of the assay system of FIG. 9, with the first foil or membrane removed.

FIG. 11 illustrates a perspective view of some components of the portion of the assay system of FIG. 7, including a second foil or membrane.

FIG. 12 illustrates the same perspective view of FIG. 11 of the same components of the portion of the assay system of FIG. 11, with the second foil or membrane removed.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with the technology have not been shown or described in detail to avoid unnecessarily obscuring descriptions of the embodiments.

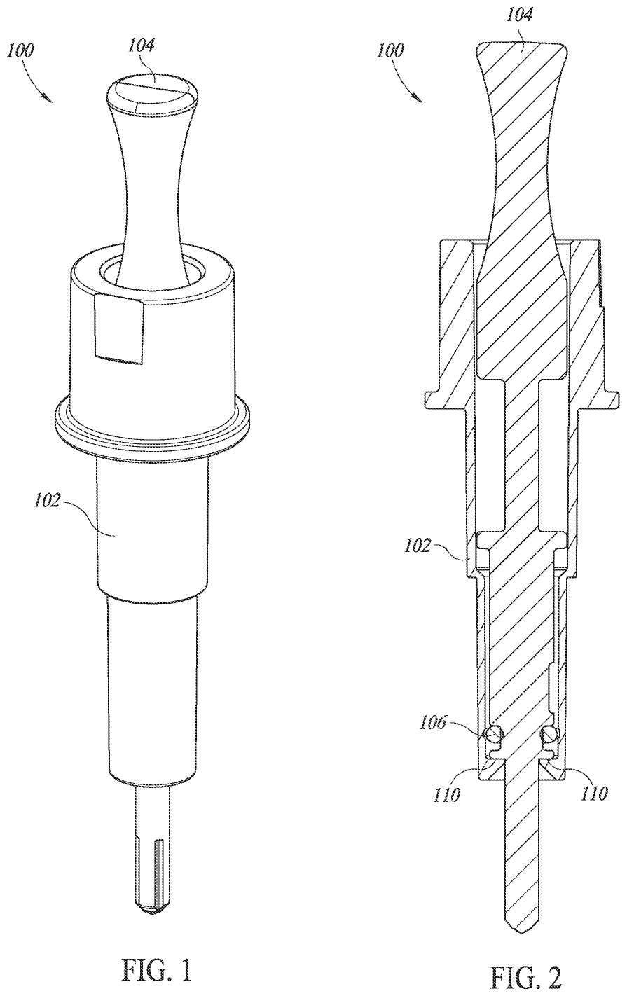

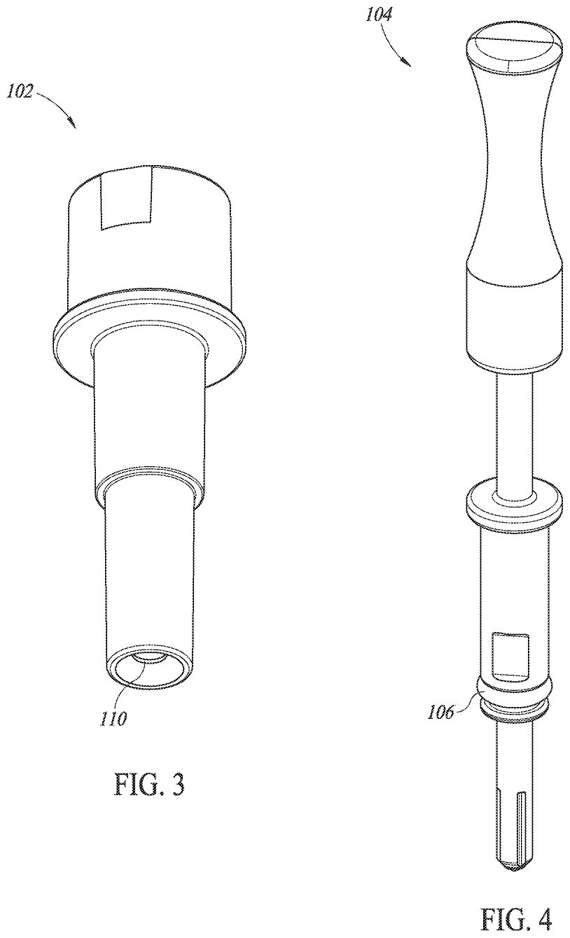

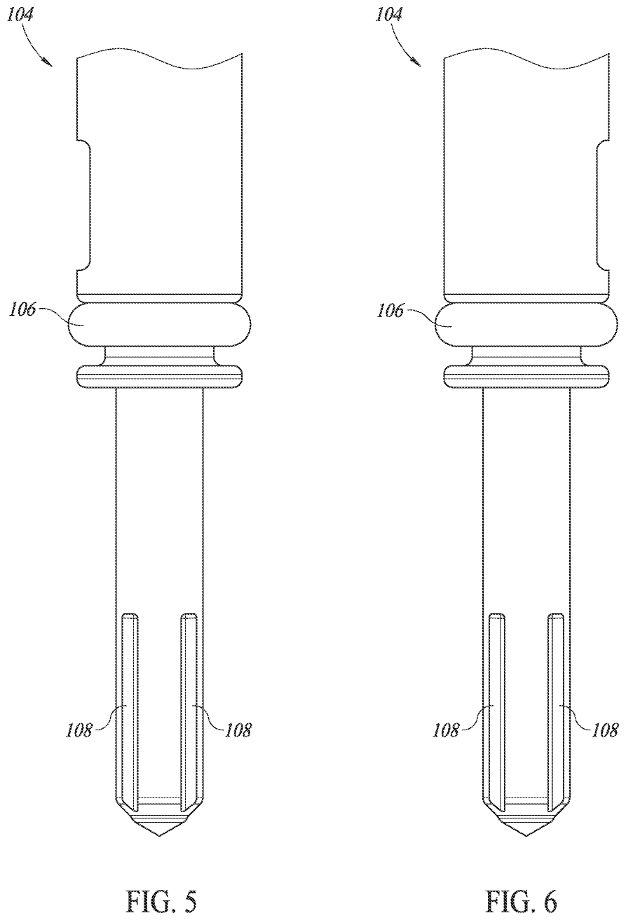

FIG. 1 illustrates a perspective view of a swab assembly 100, and FIG. 2 illustrates a cross-sectional view of the swab assembly 100. As illustrated in FIGS. 1 and 2, the swab assembly 100 includes an external metering device 102, which may be referred to as a “squeegee,” and an internal swab device 104, which can extend through the interior of the metering device 102. FIG. 3 illustrates the metering device 102 by itself, with the swab device 104 removed, and FIG. 4 illustrates the swab device 104 by itself, with the metering device 102 removed. FIGS. 5 and 6 illustrate opposing side views of a sample-collection tip end portion of the swab device 104, where the views are enlarged to improve visibility of features therein.

As illustrated in FIGS. 5 and 6 in particular, an O-ring seal 106 can be positioned on the swab device 104 adjacent to its sample-collection tip end portion. The O-ring seal 106 can provide a seal against other components, such as of an assay system. In some embodiments, the entirety of the swab device 104, other than the O-ring seal 106, can be made of a single integral, monolithic piece of injection-molded plastic material. This stands in contrast to many prior or traditional swab devices, which include a soft sample-collection tip end portion. As also illustrated in FIGS. 5 and 6 in particular, the sample-collection tip end portion of the swab device 104 includes a plurality of grooves 108 formed in an outer surface thereof. In the illustrated embodiment, the swab device 104 includes four grooves 108 that are equally spaced apart from one another circumferentially about the outer surface of the sample-collection tip end portion of the swab device 104. In alternative embodiments, however, the swab device 104 may include other numbers of grooves 108, such as one, two, three, five, six, eight, ten, twelve, or more grooves 108. Further, in alternative embodiments, the grooves 108 may not be equally spaced apart from one another.

In the illustrated embodiment, each of the grooves 108 is linear and extends along a respective axis parallel to a central longitudinal axis of the swab device 104 as a whole and to the axes of the other grooves 108. In alternative embodiments, the grooves may have other shapes and/or may follow other paths such that, for example, they are not parallel to the central longitudinal axis of the swab device 104 and/or are not parallel to one another. In the illustrated embodiment, each of the grooves 108 has a respective first end nearest to the adjacent terminal tip end of the swab device 104, and the first ends of the grooves 108 collectively lie on a single circle having a center on the central longitudinal axis of the swab device 104, and within a single plane that is perpendicular to the central longitudinal axis of the swab device 104. Thus, the first ends of the grooves 108 are equidistant from the adjacent terminal tip end of the swab device 104. In the illustrated embodiment, each of the grooves 108 has a respective second end farthest from the adjacent terminal tip end of the swab device 104, and the second ends of the grooves 108 collectively lie on a single circle having a center on the central longitudinal axis of the swab device 104, and within a single plane that is perpendicular to the central longitudinal axis of the swab device 104. Thus, the second ends of the grooves 108 are equidistant from the adjacent terminal tip end of the swab device 104.

Thus, each of the grooves 108 has the same overall length from its respective first end to its respective second end. In some embodiments, the overall length of the grooves 108 is greater than a distance from the first ends of the grooves 108 to the adjacent terminal tip end of the swab device 104. For example, the length of the grooves 108 can be at least two times, three times, or four times as long as the distance from the first ends of the grooves 108 to the adjacent terminal tip end of the swab device 104. In some embodiments, the grooves 108 can have widths or thicknesses of greater than 0.1 mm, 0.2 mm, 0.3 mm, or 0.4 mm, and/or less than 0.6 mm, 0.7 mm, 0.8 mm, or 0.9 mm, or of about 0.5 mm. In some embodiments, the grooves 108 can have depths of greater than 0.1 mm, 0.2 mm, or 0.3 mm, and/or less than 0.4 mm, 0.5 mm, 0.6 mm, or 0.7 mm, or of about 0.34 mm. In some embodiments, the grooves 108 can have lengths of greater than 1.0 mm, 2.0 mm, 3.0 mm, or 4.0 mm, and/or less than 5.0 mm, 6.0 mm, 7.0 mm, or 8.0 mm, or of about 4.52 mm.

As illustrated in FIGS. 1 and 2 in particular, the swab assembly 100, including the metering device 102 and the swab device 104, can be provided as an assembled unit, with the swab device 104 positioned within, and extending through, a cavity within the metering device 102. The metering device 102 includes an annular blade portion 110 which is engaged with an outer surface of the sample-collection tip end portion of the swab device 104, such as in an interference fit, such that the annular blade portion 110 is in physical contact and engaged with, and is pressed against, the outer surface of the sample-collection tip end portion of the swab device 104.

In practice, a patient, physician, or other medical professional or user or operator of the swab assembly 100 can insert the sample-collection tip end portion of the swab device 104 into a patient's nostril, and rotate the sample-collection tip end portion of the swab assembly 100 several (e.g., five) times inside the patient's nostril, to collect a biological sample on the swab assembly 104. The user or operator can then insert the sample-collection tip end portion of the swab device 104 into the patient's other nostril, and rotate the sample-collection tip end portion of the swab assembly 100 several (e.g., five) times therein, to collect additional biological sample on the swab assembly 104. Once the sample has been collected on the sample-collection tip end portion of the swab assembly 100, the user or operator can remove the swab device 104 from the metering device 102 by pulling the swab device 104 through the metering device 102 (that is, by pulling the swab device 104 upward relative to the metering device 102 in the orientation illustrated in FIG. 2).

As the user or operator removes the swab device 104 from the metering device 102 in this manner, the annular blade portion 110 of the metering device 102 slides over the length of the sample-collection tip end portion of the swab device 104. In doing so, given the engagement between annular blade portion 110 of the metering device 102 and the outer surface of the sample-collection tip end portion of the swab device, the metering device 102 wipes away sample collected on the external surface of the sample-collection tip end portion of the swab device 104, but leaves behind sample held in the grooves 108. In this sense, the metering device 102 acts as a squeegee, and meters the volume of the sample collected and remaining on the sample-collection tip end portion of the swab device 104. For example, the volume of the sample collected and remaining for further assay may be between 0.01 and 1.00 microliters. Put another way, the metering device 102 can be used to ensure that the volume of the sample collected and remaining for further assay conforms to a relatively narrow band of volumes (e.g., two orders of magnitude), which is narrower than many existing or traditional swab devices having soft sample-collection tip end portions, and can be advantageous in subsequent assays.

In some embodiments, the metering device 102 and/or the swab device 104 may be made, partially or completely, of hard plastic materials and/or flexible or elastic materials. In the illustrated embodiment, the sample-collection tip end portion of the swab device 104 has a circular or cylindrical cross-sectional profile. In alternative embodiments, this profile can have other shapes, including oval, elliptical, triangular, square, etc. Such shapes can be selected to improve performance in terms of sample collection, comfort, metering, etc.

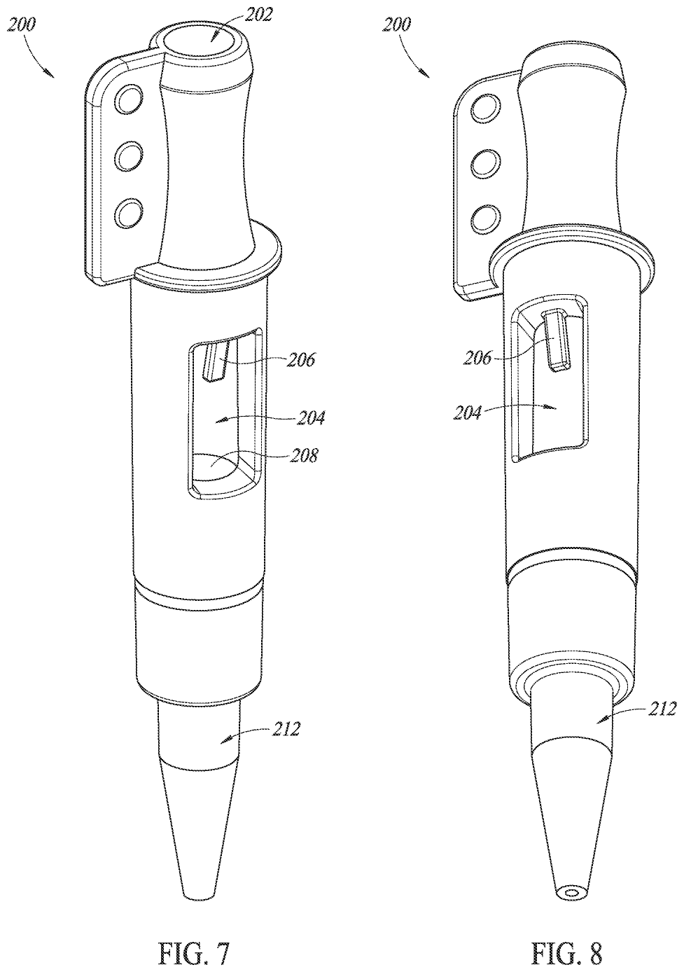

FIGS. 7 and 8 illustrate perspective views of an assay system 200 configured to receive the swab device 104. As illustrated in FIGS. 7 and 8, the assay system 200 has an overall cylindrical shape and a first opening or aperture 202 at a first end thereof along a central longitudinal axis thereof, through which the swab device 104 can be inserted longitudinally into the assay system 200. As also illustrated in FIGS. 7 and 8, the assay system 200 includes a second opening or window 204 that extends through a sidewall of the assay system 200, and through which implements can be inserted into the assay system 200, such as to operate on the assay system 200 and/or the swab device 104 therein. As further illustrated in FIGS. 7 and 8, the assay system 200 includes a protrusion, a clip, a finger portion, or a locking tab 206. The locking tab 206 extends from a first sidewall of the window 204, proximal to the aperture 202, into the window 204, along an axis substantially aligned with the central longitudinal axis of the assay system 200, and extends slightly radially inward into the interior of the assay system 200.

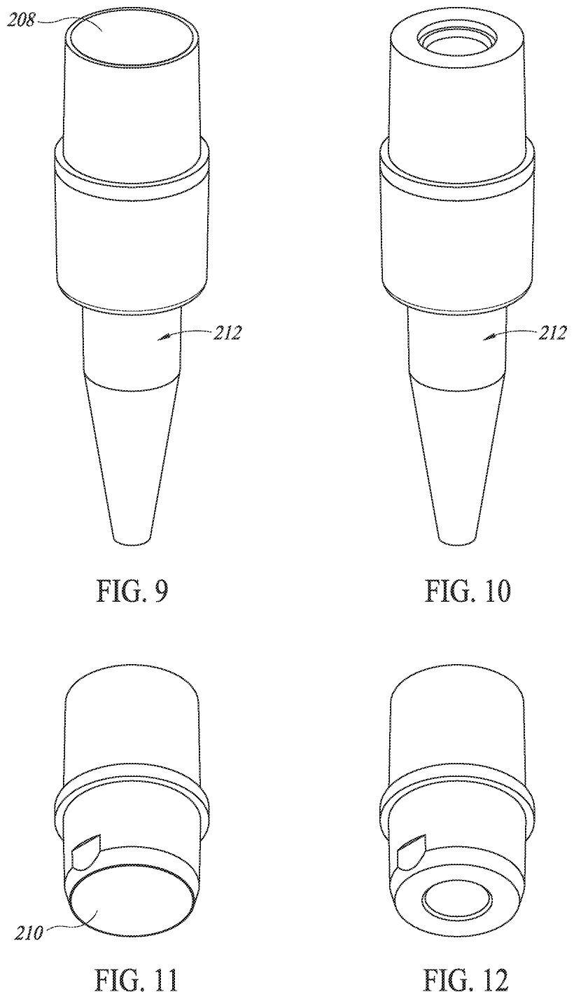

FIGS. 9 and 10 illustrate that the assay system 200 includes a first foil or membrane 208 proximal to the aperture 202 through which the swab device 104 is inserted into the assay system 200. FIGS. 11 and 12 illustrate that the assay system 200 includes a second foil or membrane 210 distal to the aperture 202 through which the swab device 104 is inserted into the assay system 200 (relative to the first foil or membrane 208). The assay system 200 may have a first internal reaction chamber located between and sealed from an external environment by the first and second foils or membranes 208, 210, and a second internal reaction chamber located within a tip end 212 of the assay system 200 distal to the aperture 202 through which the swab device 104 is inserted into the assay system 200. Each of these chambers can house various reagents or other materials useful in performing assays on the biological sample collected on the sample-collection tip end portion of the swab device 104.

In use, once the user or operator has removed the swab device 104 from the metering device 102, the user or operator can insert the sample-collection tip end portion of the swab device 104 longitudinally into the assay system 200 through the aperture 202. As the swab device 104 is inserted longitudinally into the assay system 200 through the aperture 202 in this manner, the swab device 104 comes into contact with the locking tab 206, temporarily displacing the locking tab 206 radially outward as wider portions of the swab device 104 pass by the locking tab 206, and allowing the locking tab 206 to return radially inward as narrower portions of the swab device 104 pass by the locking tab 106. As this occurs, the user will be provided with tactile feedback by action of the locking tab 206 interacting with the swab device 104. The operator may press the swab device 104 into the assay system until an end surface of the swab device 104 is flush with an end surface of the assay system 200. When the swab device 104 is fully inserted into the assay system 200 in this manner, the locking tab 206 engages with a flange or other mechanical component of the swab device 104 to prevent straightforward retraction of the swab device 104 out of the assay system 200 through the aperture 202. Furthermore, as the swab device 104 is inserted in this manner, the sample-collection tip end portion of the swab device 104 can pierce the first foil or membrane 208 and introduce the biological sample into reagents in the first internal reaction chamber for reaction and assay therein.

The user or operator can insert the assay system 200 into a base station for further operation, testing, and reactions and/or assay steps. Once the assay system 200 has been inserted into a base station, the swab device 104 can be manipulated through the window 204 and pushed further along the central longitudinal axis of the assay system 200. For example, in some embodiments, the user or operator can extend a finger or a tool into the assay system 200 through the window 204 to push the swab device 104 further along the central longitudinal axis of the assay system 200. In other embodiments, the base station may include a stepper motor and an actuator controlled by the stepper motor that is configured to engage with the swab device 104 through the window 204 and push the swab device 104 further along the central longitudinal axis of the assay system 200. As the swab device 104 is pushed in this manner, the sample-collection tip end of the swab device 104 can pierce the second foil or membrane 210 and introduce the biological sample or products thereof into reagents in the second internal reaction chamber for further reaction and assay therein.

In the illustrated embodiments, the metering device 102 and its annular blade portion 110 are components of the swab assembly 100. In alternative embodiments, however, the metering device 102 and/or its annular blade portion 110 can be components of the assay system 200. In such embodiments, the metering device 102 and/or the annular blade portion 110 can remain static while the swab device 104 and the sample-collection tip end portion of the swab device 104 is pulled through the metering device 102 and/or the annular blade portion 110. In such embodiments, movement of the sample-collection tip end portion of the swab device 104 through the annular blade portion 110 can be automated and/or controlled by the base station, such as by a stepper motor, and the metering device 102 and/or its annular blade portion 110 may be made of elastic materials.

Aspects of the various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A swab assembly, comprising:
a swab device; and
a metering device;
wherein the swab device extends through an interior of the metering device.

2. The swab assembly of claim 1 wherein the swab device is made of a single monolithic piece of injection-molded plastic.

3. The swab assembly of claim 1 wherein the swab device has a sample-collection tip end portion and a plurality of grooves formed in an outer surface of the sample-collection tip end portion.

4. The swab assembly of claim 3 wherein the plurality of grooves includes four grooves that are equally spaced apart from one another circumferentially about the outer surface of the sample-collection tip end portion.

5. The swab assembly of claim 3 wherein each of the grooves is linear and aligned with a central longitudinal axis of the swab device.

6. The swab assembly of claim 3 wherein each of the grooves is longer than a distance from the groove to a terminal tip end of the swab device.

7. The swab assembly of claim 3 wherein each of the grooves has a width of about 0.5 mm, a depth of about 0.34 mm, and a length of about 4.52 mm.

8. The swab assembly of claim 1 wherein the metering device includes an annular blade portion engaged with an outer surface of a sample-collection tip end portion of the swab device.

9. The swab assembly of claim 8 wherein the annular blade portion is engaged with the outer surface of the sample-collection tip end portion of the swab device in an interference fit.

10. An assay system configured to receive a swab device, comprising:
a generally cylindrical body;
an open window extending through a side wall of the generally cylindrical body; and
a locking tab extending from the generally cylindrical body partially through the open window;
wherein the locking tab is configured to engage with the swab device to prevent retraction of the swab device out of the assay system.

11. The assay system of claim 10 wherein the locking tab extends along an axis substantially aligned with a central longitudinal axis of the assay system and extends at least partially inward into an interior of the generally cylindrical body.

12. The assay system of claim 10, further comprising:
a first foil;
wherein, when the swab device is inserted into the assay system, the swab device pierces the first foil.

13. The assay system of claim 12, further comprising:
a second foil;
wherein the window is configured to receive a tool configured to interact with the swab device while the swab device is inside the generally cylindrical body to move the swab device and wherein, when the swab device is moved by the tool, the swab device pierces the second foil.

14. The assay system of claim 13 wherein the tool is configured to be controlled by a stepper motor.
